(19) United States
(12) Patent Application Publication
(10) Pub. No.: US 2025/0195122 A1
(43) Pub. Date: Jun. 19, 2025

(54) ELECTROSURGICAL DEVICES AND SYSTEMS

(71) Applicant: Relign Corporation, CAMPBELL, CA (US)

(72) Inventors: Aaron Germain, San Jose, CA (US); Pedro Dominguez Ramos, Campbell, CA (US)

(21) Appl. No.: 18/844,260

(22) PCT Filed: Mar. 22, 2023
(86) PCT No.: PCT/US2023/015923

§ 371 Date: Sep. 5, 2024

Related U.S. Application Data
(60) Provisional application No. 63/323,812, filed on Mar. 25, 2022.
(60) Provisional application No. 63/342,827, filed on May 17, 2022.

(51) Int. Cl. A61B 18/00; A61B 90/30

(52) U.S. Cl. CPC A61B 18/00; A61B 90/30; A61B 2018/00166; A61B 2018/00184; A61B 2018/00589; A61B 2018/00601; A61B 2018/00607; A61B 2018/00744; A61B 2218/002

(57) ABSTRACT

A probe for an electrosurgical device for treating tissue, the probe optionally including any one or combination of: an elongated shaft having a. proximal end, a distal end, and a longitudinal axis; a first electrode selectively extending from the distal end of the shaft; a second electrode extending from the distal end of the shaft, wherein the second electrode is spaced, from the first electrode; and an apron positioned at the distal end, wherein the apron is positioned between the first electrode and the tissue, and wherein the apron is positioned between the second electrode and the tissue.

### CLAIM OF PRIORITY

This application claims the benefit of U.S. Provisional Patent Application Ser. No. 63/323,812, filed on Mar. 25, 2022, and claims the benefit of U.S. Provisional Patent Application Ser. No. 63/342,827, filed on May 17, 2022, the benefit of priority of each of which is claimed hereby, and each of which is incorporated by reference herein in its entirety.

### FIELD OF THE INVENTION

The present invention relates generally to apparatuses for surgical procedures such as a total joint arthroplasty and, more particularly, to apparatuses to surgically treat tissue such as bone and soft tissue.

### BACKGROUND

A variety of surgical apparatuses exist for endoscopic cutting and removal of bone including for subacromial decompression, anterior cruciate ligament reconstruction involving notchplasty, and arthroscopic resection of the acromioclavicular joint. Currently, surgeons use arthroscopic shavers and burrs having rotational cutting surfaces to remove hard tissue in such procedures.

To promote efficiency, endoscopic tool systems including a reusable handpiece and a selection of interchangeable tool probes having different working ends are available. Individual working ends may each have two or more functionalities, such as soft tissue removal and hard tissue resection, fluid removal and imaging, so such tools systems can provide dozens of specific functionalities, providing great flexibility. Typically multiple different tools must be utilized during surgery including different tools to perform coagulation and cutting. This is particularly the case with total joint arthroplasty procedures where multiple tools with different functionality are used.

### Overview

There is a need for a single tool system to accommodate various functions. However, design of such a single tool system is a challenge. Many endoscope probes in use include lumens within rotatable shafts for the vacuum aspiration of fluids including tissue debris from the working site. These tools also rely on the delivery of radiofrequency (RF) current from the handpiece to the working end of the probe for coagulation or ablation. The interface between the reusable handpiece and the replaceable (usually disposable) probe must be designed to allow mechanical and electrical connection while managing fluid exchange so that the tools may be operated without electrical shorting.

The present inventor has developed improved surgical apparatuses and systems, such as with the combined capability to perform coagulation, endoscopic tissue cutting using RF and fluid removal. The present inventor has also developed a surgical apparatus and system with further capabilities including an apparatus and system that can illuminate the surgical site, can remove smoke in addition other tissue debris generated from the coagulation, cutting or other processes, provide irrigation, etc. The present inventor further has developed a system wherein a reusable or other handpiece may be removably connected to the replaceable, usually disposable, probe while permitting the various functions discussed above while allowing for vacuum aspiration of fluids including tissue debris through a probe shaft and outwardly through the handpiece without interfering with the electrical and/or mechanical operation of the surgical system to deliver radiofrequency (RF) current to the probe. The present inventor contemplates the surgical apparatuses and systems can reduce costs by eliminating multiple surgical tools, reduce surgical complexity and reduce surgical time among other benefits. The probes and or electrosurgical devices disclosed herein can be configured to be reusable or can be configured to be disposable after the surgery.

2. Description of the Background Art. Relevant commonly owned patent publications include: US 2018-0303509; US 2019-0008541; US 2019-0059983; US 2019-0134279; US 2019-0021788; US 2018-0317957; US 2019-0008538; US 2019-0083121; US 2018-0263649; and US 2019-0015151, the full disclosures of which are incorporated herein by reference.

The following, non-limiting examples, detail certain aspects of the present subject matter to solve the challenges and provide the benefits discussed herein, among others.

Example 1 is a probe for an electrosurgical device for treating tissue, the probe optionally including any one or combination of: an elongated shaft having a proximal end, a distal end, and a longitudinal axis; a first electrode selectively extending from the distal end of the shaft; a second electrode extending from the distal end of the shaft, wherein the second electrode is spaced from the first electrode; and an apron positioned at the distal end, wherein the apron is positioned between the first electrode and the tissue, and wherein the apron is positioned between the second electrode and the tissue.

Example 2 is the probe of Example 1, wherein optionally the apron is a flexible non-conductive material.

Example 3 is the probe of any one or any combination of Examples 1-2, wherein optionally the apron is a synthetic elastomeric polymer material.

Example 4 is the probe of any one or any combination of Examples 1-3, wherein optionally the apron is transparent.

Example 5 is the probe of any one or any combination of Examples 1-4, wherein optionally the apron substantially surrounds and encloses all but a tip of the first electrode and all but a tip of the second electrode.

Example 6 is the probe of any one or any combination of Examples 1-5, wherein optionally the apron is removable from at least a portion of one of the first electrode and the second electrode.

Example 7 is the probe of any one or any combination of Examples 1-6, optionally further including a plurality of tubes extending along the shaft to the distal end, wherein the first electrode is connected to at least a first of the plurality of tubes and has a channel therein extending to a first port defined by the first electrode, and wherein the second electrode is connected to at least a second of the plurality of tubes and has a channel therein extending to a first port defined by the first electrode.

Example 8 is the probe of any one or any combination of Examples 1-7, wherein optionally at least a first portion of the second electrode is retractable relative to one or both of the first electrode and a second portion of the second electrode.

Example 9 is the probe of any one or any combination of Examples 1-8, wherein optionally the first portion of the second electrode is longitudinally retractable into the shaft.

Example 10 is the probe of any one or any combination of Examples 1-9, wherein optionally the apron is moveable with retraction of the first portion of the second electrode to expose at least one of a second portion of the second electrode and the first electrode.

Example 11 is the probe of any one or any combination of Examples 1-10, wherein optionally retraction of the first portion of the second electrode changes a surface area of the second electrode exposed to the tissue.

Example 12 is the probe of any one or any combination of Examples 1-11, wherein optionally one of the first electrode has a different shape from the second electrode or the first electrode has a substantially identical shape as the second electrode.

Example 13 is the probe of any one or any combination of Examples 1-12, optionally further including an illumination device positioned at the distal end of the shaft.

Example 14 is the probe of any one or any combination of Examples 1-13, wherein optionally the illumination device is positioned within the apron.

Example 15 is the probe of any one or any combination of Examples 1-14, optionally further including a handle, wherein the probe is configured to couple with the handle.

Example 16 is the probe of any one or any combination of Examples 1-15, wherein optionally the apron extends between the first electrode and the second electrode and has an opening at a distal side.

Example 17 is the probe of any one or any combination of Examples 1-16, wherein optionally the first electrode and the second electrode have a first configuration relative to one another in an electrosurgical coagulation mode.

Example 18 is the probe of any one or any combination of Examples 1-17, wherein optionally the first electrode and the second electrode have a second configuration relative to one another in an electrosurgical resection mode.

Example 19 is the probe of any one or any combination of Examples 1-18, wherein optionally the apron is configured to contain a liquid at a surface of the tissue adjacent the first electrode and the second electrode, wherein the liquid is inflowed through a passage in one of the first electrode or the second electrode, and wherein the liquid is suctioned from a cavity defined at least partially by the apron by a passage in the other of the first electrode or second electrode.

Example 20 is the probe of any one or any combination of Examples 1-19, wherein optionally wherein a first portion of the second electrode is selectively moveable thereby reducing a surface area of the second electrode exposed to the tissue.

Example 21 is the probe of any one or any combination of Examples 1-20, wherein optionally the first portion of the second electrode represents no less than at least 90%, or 80% or 70% of a total surface area of the second electrode.

Example 22 is the probe of any one or any combination of Examples 1-21, wherein optionally a second portion of the second electrode that remains stationary is no less than at least 10%, 20% or 30% of the total surface area of the second electrode.

Example 23 is the probe of any one or any combination of Examples 1-22, wherein optionally a surface area of the first electrode is no more than at least twice as large as a surface area of the second portion of the second electrode.

Example 24 is the probe of any one or any combination of Examples 1-23, wherein optionally a surface area of the first electrode is configured to generate a thermal effect in an electrosurgical coagulation mode and substantially no thermal effect in an electrosurgical resection mode.

Example 25 is the probe of any one or any combination of Examples 1-24, optionally further including a spring coupled to the first electrode, wherein the spring is configured to allow the first electrode to be longitudinally movable to contour with the tissue during electrosurgical resection mode.

Example 26 is the probe of any one or any combination of Examples 1-25, wherein optionally the first electrode is stationary during electrosurgical coagulation mode and movable during electrosurgical resection mode.

Example 27 is the probe of any one or any combination of Examples 1-26, wherein optionally at least a first portion of the second electrode is moveable and movement of the first portion of the second electrode engages the first electrode with a spring, wherein the spring is configured to allow the first electrode to be longitudinally movable.

Example 28 is a probe for treating tissue, the probe optionally including any one or any combination of: an elongated shaft having a proximal end, a distal end, and a longitudinal axis; a plurality of tubes extending along the shaft to the distal end; a first electrode positioned adjacent the distal end, wherein the first electrode is rotatably coupled to one of the shaft or a first of the plurality of tubes; and a second electrode positioned adjacent the distal end and spaced from the first electrode, wherein the second electrode is rotatably coupled to one of the shaft or a second of the plurality of tubes; wherein a third of the plurality of tubes extends distal of the distal end of the shaft and is positioned between the first electrode and the second electrode.

Example 29 is the probe of Example 28, wherein optionally the first electrode and the second electrode are configured to roll over the tissue with movement of the probe relative to the tissue.

Example 30 is a probe for treating tissue, the probe optionally including any one or combination of: an elongated shaft having a proximal end, a distal end, and a longitudinal axis; a first electrode positioned adjacent the distal end; and a second electrode positioned adjacent the distal end, wherein the second electrode is spaced from the first electrode; wherein at least a first portion of the second electrode is retractable relative to the first electrode.

Example 31 is the probe of Example 30, optionally further including an apron positioned at the distal end, the apron positioned between the first electrode and the tissue and positioned between the second electrode and the tissue.

Example 32 is the probe of any one or any combination of Examples 30-31, wherein optionally the apron is movable with retraction of the first portion of the second electrode to expose a second portion of the second electrode to the tissue.

Example 33 is the probe of any one or any combination of Examples 30-32, wherein optionally the first portion of the second electrode is longitudinally retractable into the shaft.

Example 34 is the probe of any one or any combination of Examples 30-33, wherein optionally retraction of the first portion of the second electrode changes a surface area of the second electrode exposed to the tissue.

Example 35 is the probe of any one or any combination of Examples 30-34, wherein optionally the second electrode has a substantially identical shape as the first electrode prior to retraction of the first portion of the second electrode.

In Example 36, the devices or systems of any one or any combination of Examples 1-35 can optionally be configured such that all elements or options recited are available to use or select from.

### BRIEF DESCRIPTION OF THE DRAWINGS

Various embodiments of the present invention will now be discussed with reference to the appended drawings. It should be appreciated that the drawings depict only typical embodiments of the invention and are therefore not to be considered limiting in scope.

FIG. 1 is an exploded view of an arthroscopic cutting system that includes an electrosurgical device having reusable handpiece with a detachable single-use probe according to an example of the present disclosure.

FIG. 2 is a perspective view of the electrosurgical device of FIG. 1 with the handpiece coupled to the probe according to an example of the present disclosure.

FIG. 3 is an enlarged perspective view of a distal (working) end of the probe of FIG. 2 showing various components thereof according to an example of the present disclosure.

FIG. 4 is an enlarged perspective view of the distal (working) end of a probe with an apron according to another example of the present disclosure.

FIG. 4A is a schematic view of the distal end of the probe with the apron of FIG. 4 according to an example of the present disclosure.

FIG. 4B is a schematic view of the distal end of the probe with the apron interposed between portions of the electrodes according to an example of the present disclosure.

FIG. 5 is an enlarged perspective view of the distal (working) end of a probe according to another example with a portion of a first electrode retracted a first distance relative to a second electrode according to an example of the present disclosure.

FIG. 6 is an enlarged perspective view of the distal (working) end of the probe according to another example with a portion of the first electrode retracted a second distance relative to a second electrode according to an example of the present disclosure.

FIG. 7 is an enlarged perspective view of the distal (working) end of the probe with the apron such as that of FIGS. 4-4B with a second electrode retracted to remove the apron from covering a first electrode according to an example of the present disclosure.

FIG. 8A is a cross-sectional view of a hub of the probe of FIGS. 1 and 2 taken along showing an actuation mechanism for retracting and extending the first electrode with the actuation mechanism in a first position.

FIG. 8B is a cross-sectional view of the hub and the actuation mechanism of FIG. 8A with the actuation mechanism in a second position.

FIG. 9 is an enlarged perspective view of the distal (working) end of a probe similar to the one shown in FIG. 7 with a portion of a second electrode this is retractable relative to a first electrode and/or the distal end according to an example of the present disclosure.

FIG. 10 is a perspective view of the distal (working) end of the probe of FIG. 9 with the portion of the first electrode retracted relative to the second electrode and/or the distal end according to an example of the present disclosure.

FIG. 11 shows an electrosurgical device with a probe and a handpiece having an actuator for moving the actuation mechanism for retracting and extending the first electrode as described in FIGS. 5-10.

FIGS. 12A and 12B show side views of an electrosurgical device with a probe and a handpiece with the probe having electrodes configured as rolling elements according to an example of the present disclosure.

FIG. 13 is a schematic view of the distal (working) end of the probe of FIGS. 12A and 12B with first and second electrodes configured as the rolling elements.

FIG. 14A is an enlarged perspective view of the distal (working) end of a probe similar to the one shown in FIG. 9 operating in a coagulation mode.

FIG. 14B is the probe of FIG. 14A with the portion of the first electrode retracted relative to the second electrode and/or the distal end to operate in an ablation/tissue cutting mode according to an example of the present disclosure.

FIG. 15 is an enlarged perspective view of the distal (working) end of a probe with a suction port at or adjacent the distal end of the shaft for coagulation mode according to an example of the present application.

FIG. 16 is a schematic view of the distal end of the probe of FIG. 15 showing flow and suction during coagulation mode according to an example of the present disclosure.

FIG. 17 is a perspective view of the distal (working) end of the probe of FIGS. 15 and 16, with the portion of the first electrode retracted relative to the second electrode and/or the distal end for ablation mode according to an example of the present disclosure.

FIG. 18 is a schematic view of the distal end of the probe of FIG. 15 showing flow and suction during ablation mode according to an example of the present disclosure.

FIGS. 19A and 19B show plan views of a distal (working) end of a probe according to another example with the suction port at or adjacent the distal end of the shaft.

FIG. 20 shows a heat map capturing a temperature profile of saline and products of coagulation during operation of the distal (working) end of the probe of FIGS. 19A and 19B according to an example of the present application.

FIG. 21 shows perspective views of an electrosurgical device operating in a coagulation mode (viewer's left) to an ablation mode (viewer's right) according to an example of the present application.

FIGS. 22-24 are enlarged views of the distal (working) end of the electrosurgical device of FIG. 21 in the coagulation mode.

FIGS. 25-27 are enlarged views of the distal (working) end of the electrosurgical device of FIG. 21 in the ablation mode.

FIGS. 28A and 28B show components internal to a shaft of the electrosurgical device including push rods for actuating electrodes according to an example of the present application.

FIG. 29 shows components internal to the electrosurgical device including an actuation mechanism, slider and hub that engage and move the push rods as desired according to an example of the present application.

### DETAILED DESCRIPTION OF THE INVENTION

The present invention relates to electrosurgical devices that have various functions. Several embodiments of the devices will now be described to provide an overall understanding of the principles of the form, function and methods of use. In general, the present disclosure provides for electrosurgical devices that can be used as arthroscopic tools including for total joint arthroplasty. In most cases, the electrosurgical devices described herein can perform more than one surgical function. Thus, the electrosurgical devices can be configured for coagulation and/or cutting bone such as of soft tissue, meniscal tissue, etc. using RF energy. The arthroscopic tools are typically disposable and are configured for detachable coupling to a non-disposable handpiece. This description of the general principles of this invention is not meant to limit the inventive concepts in the appended claims.

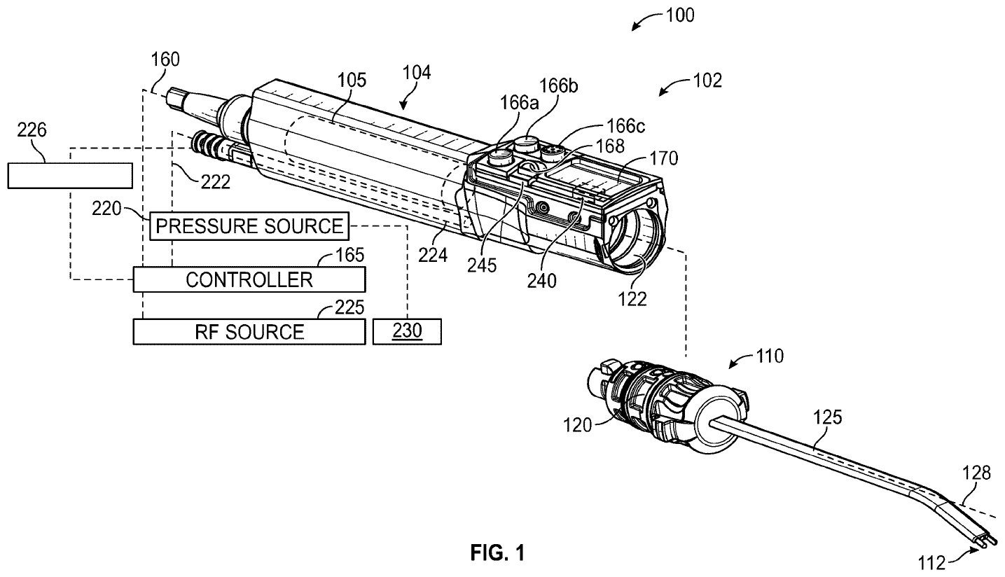

In one example shown in FIG. 1, an arthroscopic system 100 of the present invention provides an electrosurgical apparatus 102 having a handpiece 104 with motor drive 105 and a probe 110 with a proximal hub 120 that can be received by receiver or bore 122 in the handpiece 104. In one aspect, the probe 110 has a working or distal end 112 that carries RF electrodes configured for use in many arthroscopic surgical applications, including but not limited to treating bone in shoulders, knees, hips, wrists, ankles and the spine.

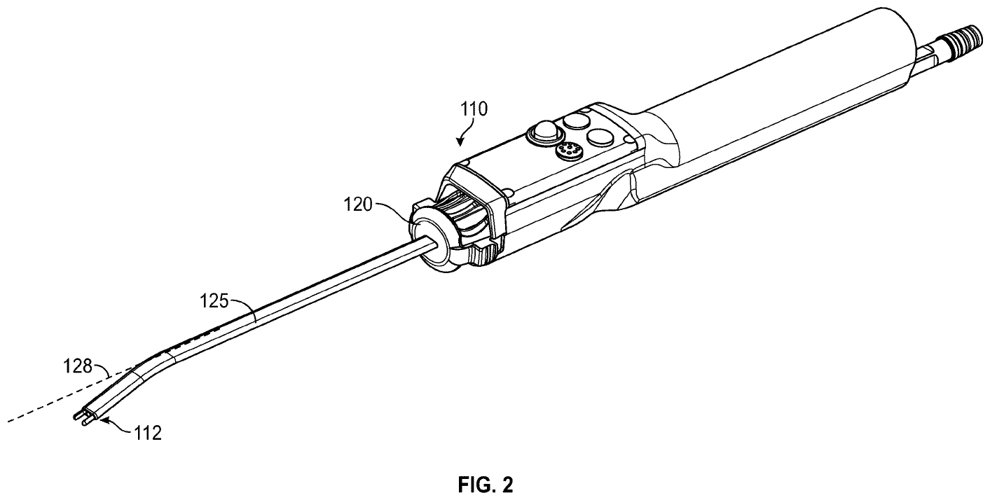

As can be seen in FIGS. 1 and 2, the probe 110 is attachable to and detachable from the handpiece 104. In FIGS. 1 and 2, the probe 110 has a shaft 125 extending along longitudinal axis 128. A distal portion of the shaft 125 including the distal end 112 can be angled (e.g., by 15 degrees, or the like) relative to the longitudinal axis 128. The shaft 125 can be somewhat flexible or rigid as desired and can house various components that can extend from the hub 120 to the distal end 112 as further discussed. Thus, the shaft 125 can comprise tube or outer sleeve with components such as wires, flow channels, additional shafts, and the like passing therethrough. The shaft 125 extends from the hub 120 (located at a proximal end of the shaft 125) to the distal end 112. The shaft 125 can be coupled in a fixed manner to the hub 120 which can be an injection molded plastic, for example, with the shaft 125 insert molded therein. One or more components can pass through the shaft 125 including to provide RF energy to the electrodes, provide for fluid removal, provide for fluid application, provide for illumination or the like. The motor drive 105 need not be utilized for some of the functions of the electrosurgical device 102 with the probe 110.

In FIG. 1, it can be seen that the handle 104 is operatively coupled by electrical cable 160 to a controller 165 which can control the motor drive unit 105, communication with a pressure source 220, and communication with the RF source 225. Actuator buttons 166a, 166b, 166c, etc. on the handpiece 104 (sometimes called a handle herein) can be used to select operating modes, such as current strength for RF, motor speed, flow control, illumination control or the like. In one variation, a joystick 168 can be moved forward and backward to adjust the rotational speed of motor or other function such as to extend or retract an electrode (discussed subsequently). Motor speed can continuously adjustable, or can be adjusted in increments. An LCD screen 170 can provided in the handpiece 104 for displaying operating parameters, such as mode of operation, etc.

It can be understood from FIG. 1 that the system 100 and handpiece 104 can be configured for use with various disposable probes which can be designed for various different functions and procedures. Some of the probes can utilize the motor drive 105, for example, and some may not. These probes are various described in the various applications incorporated by reference with the U.S. Application Publications noted above.

FIG. 1 further shows that the system 100 also includes a pressure source 220 such as a negative pressure source coupled to aspiration tubing 222 which communicates with a flow channel 224 in handpiece 104 and can cooperate with one or more tubes of the probe 110. The system 100 includes the RF source 225 which can be connected to an electrode arrangement of the probe 110. The system 100 can include flow inducing device 226 such as a pump, positive pressure source or the like that passes in fluid communication to the handpiece 104 and to the distal end 112. The flow inducing device 226 (optionally controlled by the controller 165) can allow for flow to the distal tip 112 of a fluid such as for application of an irrigating fluid (e.g., saline) utilized during operation of the electrosurgical apparatus 102. The controller 165 and microprocessor therein together with control algorithms are provided to operate and control all functionality, which includes controlling the motor drive 105, the RF source 225, the flow inducing device 226, illuminating device, and the negative pressure source 220 which can aspirate fluid including tissue debris to collection reservoir 230.

As can be understood from the above description of the system 100, the electrosurgical device 102 and handpiece 104, the controller 165 and controller algorithms can be configured to perform and automate many tasks to provide for system functionality. In a first aspect, controller algorithms are needed for device identification so that when any of the different probes types are coupled to handpiece 104, the controller 165 will recognize the probe type and then select algorithms for operating the motor drive 105, RF source 225, flow inducing device 226, negative pressure source 220, etc. as is needed for the particular probe. In a second aspect, the controller can be configured with algorithms that identify whether the probe is coupled to the handpiece 104 in a particular orientation relative to the handpiece, wherein each orientation requires a different subset of the operating algorithms.

Referring to FIG. 1, the handpiece 104 can carry a first Hall effect sensor 240 in a distal region of the handpiece 104 adjacent the receiving passageway 122 that receives the hub 120 of probe 110. The handpiece 104 can carry a second Hall effect sensor 245 adjacent the rotatable drive coupling 150 of the probe 110. The probe 110 can carry a plurality of magnets that interact with the Hall effect sensors 240, 245 to provide multiple control functions in cooperation with controller algorithms, including (i) identification of the type of probe coupled to the handpiece, and (ii) the orientation of the probe hub 120 relative to the handpiece 104.

The Hall sensor 240 and controller algorithms can be adapted to read the magnetic field strength of the particular magnet(s) in the probe which can be compared to a library of field strengths that correspond to particular probe types. Then, a Hall identification signal can be generated or otherwise provided to the controller 165 to select the controller algorithms for operating the identified probe, which can include parameters for operating the motor drive 105, negative pressure source 220, the flow inducing device 226, power source (e.g., for illumination and other function) and/or RF source 225 as may be required for the probe type. The Hall sensor 240 and associated algorithms look for magnetic field strength regardless of polarity to identify the probe type.

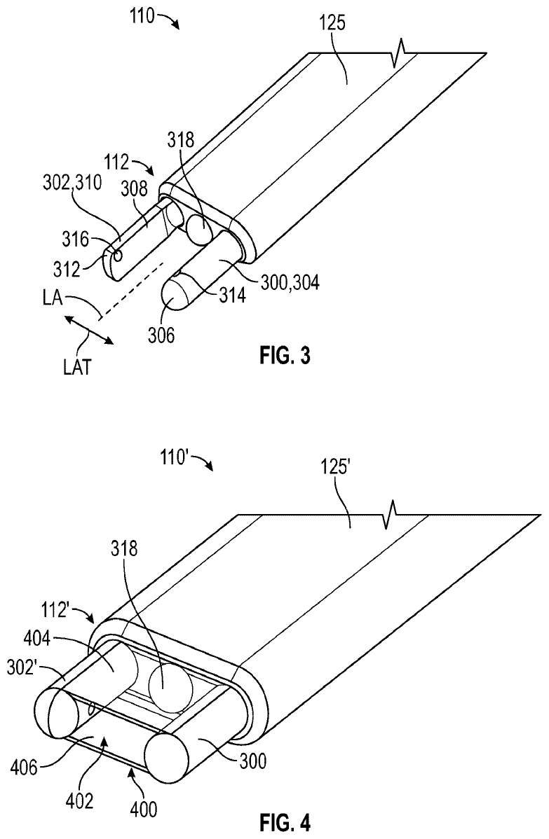

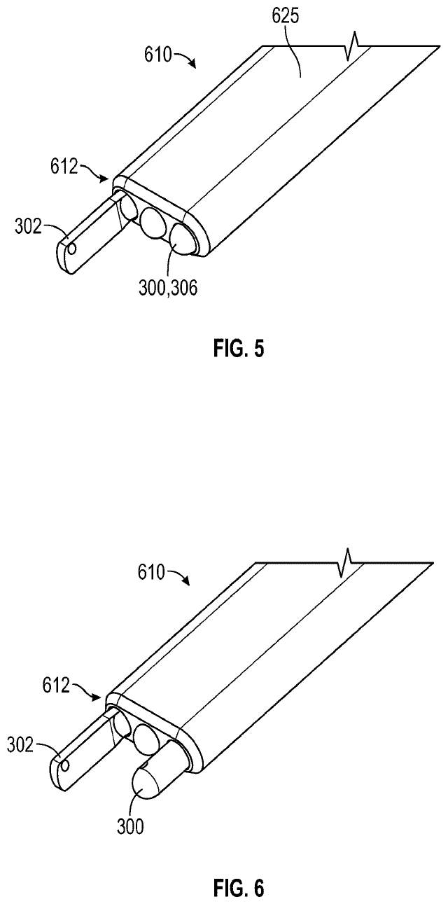

As an example, the electrosurgical device 102 can be operated in different RF modes. As described below, a one mode can deliver RF current in a cutting waveform to thereby create a plasma that ablates tissue. Such mode can be utilized with the configurations of FIGS. 5-7 and 10, for example. In another RF mode, the controller 165 can include an algorithm that utilizes both electrodes in the extended position such as shown in FIGS. 3, 4-4B, 9 and 12A-13. Then RF current in a coagulation waveform can be delivered to the electrodes. The operator can then move the stationary electrodes over a targeted site for coagulation of tissue.

FIG. 3 shows an example of the distal end 112 of the shaft 125 of the probe 110. The distal end 112 can be made of or coated with an insulating material such as ceramic, for example. A first electrode 300 can protrude from the distal end 112. Similarly, a second electrode 302 can protrude from the distal end 112. The second electrode 302 can be in a spaced apart relationship (e.g., from 0.25 mm to 25 mm, inclusive) from the first electrode 300. The first electrode 300 and second electrode 302 can have an elongate length along a longitudinal axis LA and can extend generally parallel with one another with respect to the longitudinal axis LA. The first electrode 300 and the second electrode 302 can protrude substantially a same distance from the distal end 112 (e.g., between 1 mm and 10 mm, inclusive). However, it is contemplated that the first electrode 300 could protrude a greater distance than the second electrode 302 (or vice versa) according to further examples. The first electrode 300 and the second electrode 302 can be constructed of conductive metal or metal alloy such as tungsten, alloys including tungsten, or the like. The first electrode 300 and the second electrode 302 can be configured for bipolar operation with the first and second electrode 300, 302 alternating between active and return. Level of RF energy to the first and second electrodes 300, 302 can be controlled as desired for use in coagulation or RF ablation.

The first and second electrodes 300, 302 can extend internally within the shaft 125 a distance including all the way to the hub in some cases. Alternatively, the electrodes 300, 302 can terminate such as at or just proximal of the distal end 112. As further discussed subsequently, at least one of the first and second electrodes 300, 302 can be configured to move, this can be via direct engagement of at least one of the first and second electrodes 300, 302 by an actuation mechanism or via other intermediate component(s) coupled to the actuation mechanism and the first and/or second electrodes 300, 302. For simplicity, the present disclosure uses the term “actuator element” or “second shaft” to describe not only any intermediate component(s) if utilized but the term also includes proximal portion(s) of the first electrode 300 and/or second electrode 302 themselves if directly engaged. Thus, the term “second shaft” or variations thereof can include proximal portions the first electrode 300 and/or second electrode 302 itself or other component(s) indirectly coupled to the first electrode 300 and/or second electrode 302.

The first electrode 300 can have a different shape than the second electrode 302 (and hence the surface area of the first electrode 300 can differ from the surface area of the second electrode 302). However, it is contemplated that in some examples the shape of the first electrode 300 and the second electrode 302 can be substantially the same. The first electrode 300 can have a cylindrically shaped body 304 with a semi-spherical tip 306. A diameter of the first electrode 300 can be between 0.02 inches to 0.75 inches, inclusive. The tip 306 can have another shape such as be flat, concave, convex, etc. according to further examples.

The second electrode 302 can have a shape of a blade or paddle, for example. Thus, the second electrode 302 can have one or more surfaces that are substantially flat or only slightly curved. These one or more surfaces can include a first inner surface 308 that comprises one of the major surfaces of the second electrode 302. Minor surfaces such as minor surface 310 can be much smaller dimensionally and in surface area than the first inner surface 308. The minor surface 310 can have a lateral width as measured in a lateral direction (LAT) of between 0.05 mm and 2.5 mm, for example. The second electrode 302 can have a tip 312. The tip 312 can be flat, chamfered, or otherwise shaped as desired. The shape of the tip 312 can differ from that of the tip 306 as the geometry of the first electrode 300 differs from the geometry of the second electrode 302.

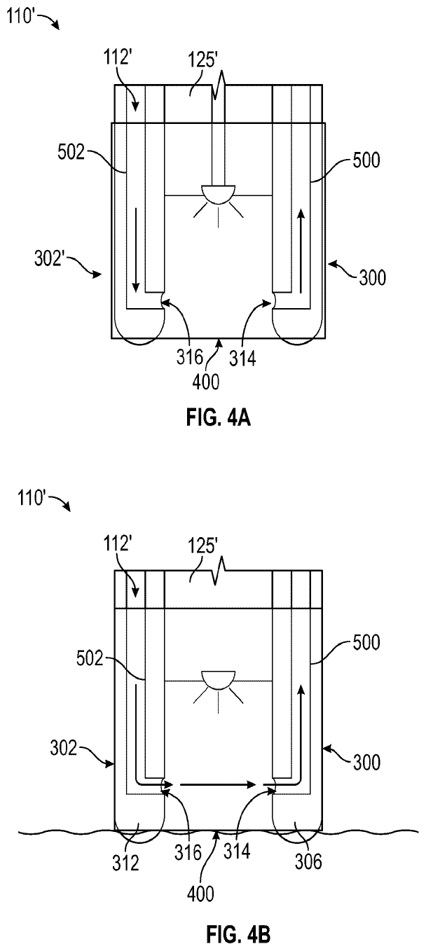

The first electrode 300 can have a port 314 therein. The port 314 can be an inlet/outlet to a first flow tube (shown subsequently such as in FIGS. 4A and 4B) that defines a flow channel. The first flow tube can be partially defined by the first electrode 300, can be in fluid communication with a flow channel through the first electrode 300 or can be an entirely separate component from the first electrode 300 that can have an outlet adjacent the first electrode 300. The first flow tube(s) can extend along the shaft 125 and can be in fluid communication with the flow channel 224 in handpiece 104 and further in communication with the aspiration tubing 222 which communicates with the negative pressure source 220 (FIG. 1). Via the port 314, the fluid (e.g., irrigating fluid, blood, tissue debris, smoke, etc.) can be aspirated and pass away from the surgical site via passing through the first electrode 300, the first flow tube, etc. The location of the port 314 in FIG. 3 is purely exemplary and can be in other locations such as at the tip 306, etc. The use of additional ports are also contemplated.

The second electrode 302 can have a second port 316 therein. This second port 316 can be located on the first inner surface 308, for example. However, other locations or additional ports or alternative port location such as those that communicate through minor surface 310, the tip 312 or another surface of the second electrode 302 is contemplated. The port 316 can be an inlet/outlet to the surgical area (also called the surgical site herein) allowing the irrigating fluid to pass from the electrosurgical device to the surgical area. The port 316 can be in fluid communication with a second flow tube (shown subsequently such as in FIGS. 4A and 4B) that defines a flow channel. The second flow tube can be partially defined by the second electrode 302 or can be in fluid communication with a flow channel through the second electrode 302. The first flow tube(s) can extend along the shaft 125 and can be in fluid communication with the flow inducing device 226 (FIG. 1).

An illumination element 318 can be positioned at or adjacent the distal end 112 such as substantially between the first electrode 300 and the second electrode 302. The illumination element 318 can be a light emitting diode (LED) or plurality of LEDs for example. The illumination element 318 can be actuated and controlled by the buttons on the handpiece, foot pedal, etc. The illumination element 318 can increase or decrease in luminance, change illumination color, etc. under control such as actuated by the buttons on the handpiece as contemplated herein. Although a single illumination element 318 is shown it is contemplated a plurality of such illumination elements can be utilized at or adjacent the distal end 112. Other locations for the single illumination element 318 such as proximal of the distal end 112 are also contemplated.

FIG. 4 shows another example of a distal end 112′ of a shaft 125′ of a probe 110′ similar to that of the probe 110 of FIGS. 1-3. In the interest of brevity, construction of the probe 110′ will not be discussed other than to focus on differences between the probe 110′ and the probe 110. The probe 110′ can include an apron 400. The apron 400 can constructed of a flexible non-conductive material such as one that is configured to prevent unintended RF conduction outside of the apron 400. For example, the material for the apron 400 can be natural or synthetic elastomeric polymer material (e.g., silicone, silicone blend, or the like). The apron 400 can be configured to elastically deform over the first electrode 300 and the second electrode 302′. The apron 400 can partially or fully encapsulate the first electrode 300, the second electrode 302′ and the space between the first electrode 300 and the second electrode 302′. The apron 400 can be sealed to or otherwise affixed to the distal end 112′. Thus, the apron 400 can be configured as a cap according to some examples fully or partially encapsulating components of the distal end 112′ including the first electrode 300 and the second electrode 302′. Put another way, the apron 400 separates at least a part(s) of the first electrode 300 from contact with the tissue and/or a part(s) of the second electrode 302′ from contact with the tissue. In the case of full encapsulation by the apron 400, only the apron 400 would be in contact with the tissue. However, FIG. 4 shows a partial encapsulation by the apron 400 with a distal opening 402 to allow part of the first electrode 300 to be directly exposed to the tissue and part of the second electrode 302′ to be directly exposed to the tissue. The apron 400 however can enclose at least some parts (such as lateral sides) of the first electrode 300 and the second electrode 302′ such that these are not in direct contact with the tissue. Due to the shape of the apron 400 and the apron 400 conforming over portions of the first electrode 300 and the second electrode 302′, the apron 400 as a first major side 404 (e.g., a top side in the view of FIG. 4) and a second major side 406 (e.g., a bottom side in the view of FIG. 4). The first major side 404 can oppose the second major side 406. However, other shapes for the apron 400 than are shown in FIG. 4 are contemplated.

The apron 400 as shown in FIG. 4 can be a separate component from the shaft 125′ and can be constructed of different material than the shaft 125′. However, it is contemplated the apron 400 could be an additional feature or part of the shaft 125′ according to some examples. It is also contemplated (and further shown in subsequent figures) that the apron 400 could have port(s) allowing for selective passage of a portion(s) of the first electrode 300 and/or the second electrode 302′ therethrough according to some examples. The apron 400 can also be designed to only cover different selective portions of the first and/or second electrodes 300, 302′ than those shown in FIG. 4. Thus, the apron 400 can be configured to allow the tip(s) of the first and/or second electrodes 300, 302′ to be exposed for direct contact with the tissue for example, while covering other surfaces of the first and/or second electrodes 300, 302′ as in the example of FIG. 4. In another example, the apron 400 could cover the tip(s) but allow for direct exposure of one or more other portion(s) of the first and/or second electrodes 300, 302′ to the tissue.

The apron 400 can be transparent or translucent according to some examples. This can aid in illuminating the surgical site including the tissue with the illumination element 318. The illumination element 318 can be positioned within the apron 400. Positioning the illumination element 318 within the apron 400 can be intentional and can provide for a lantern type illumination (duller glow) rather than a relatively brighter flashlight type illumination if the illumination element 318 was positioned outside of the apron 400. It is contemplated illuminations elements can be utilized both inside the apron 400 and outside the apron 400. The apron 400 can trap the irrigating fluid in the space between the first electrode 300 and the second electrode 302′. This can more effectively allow RF current to pass between the first and second electrodes 300, 302′. This can improve coagulation provided by the device. The apron 400 can be configured to aid in the capture and aspiration of the irrigating fluid as well as additional fluid that is the product of coagulation (e.g., coagulated blood, tissue debris, gas, smoke, etc.). Thus, the fluid can more effectively be aspirated and pass away from the surgical site with use of the apron 400.

FIGS. 4A and 4B show operation of the apron 400 and other components of the probe 110′. As shown in FIGS. 4A and 4B, the first electrode 300 can include the port 314 comprising an outlet for the fluid (e.g., irrigating fluid, blood, tissue debris, smoke, etc.) from the surgical site (e.g., as defined within and adjacent the apron 400 including in the space between the first electrode 300 and the second electrode 302′). The port 314 can be an inlet to a first flow tube 500 that defines a flow channel for the fluid. The first flow tube 500 can be partially defined by the first electrode 300 according to the example of FIGS. 4A and 4B. However, the first flow tube 500 could be a separate component according to further examples. The first flow tube 500 can extend proximally into the shaft 125′ proximal of the distal tip 112′. As discussed previously, the first flow tube 500 can extend along the shaft 125′ and can be in fluid communication with the flow channel 224 in handpiece 104 and further in communication with the aspiration tubing 222 which communicates with the negative pressure source 220 (FIG. 1).

The second electrode 302′ can have the second port 316 therein. The second port 316 can comprise outlet for irrigating fluid leaving the device from a second flow tube 502. The irrigating fluid passes through the second port 316 and enters the surgical site. The second flow tube 502 that defines a flow channel for the irrigating fluid. The second flow tube 502 can be partially defined by the second electrode 302′ according to the example of FIGS. 4A and 4B. However, the second flow tube 502 could be a separate component according to further examples. The second flow tube 502 can extend proximally into the shaft 125′ proximal of the distal tip 112′. As discussed previously, the second flow tube 502 can extend along the shaft 125′ and can be in fluid communication with the flow inducing device 226.

EF current can pass between the first electrode 300 and the second electrode 302′ within the surgical site (partially defined by the apron 400) for coagulation. FIG. 4B shows a portion of the first electrode 300 such as the tip 306 can be in direct contact with the tissue. Similarly, a portion of the second electrode 302′ such as the tip 312 can be in direct contact with the tissue. The apron 400 surrounds other portions of the surgical site and other portions of the first electrode 300 and/or the second electrode 302′. As discussed, the apron 400 can be configured help to contain irrigating fluid, smoke, etc. generated from the coagulation using RF.

FIG. 5 shows another example of a distal end 612 of a shaft 625 of a probe 610 according to another embodiment. The probe 610 has similar construct to that of the probe 110. The probe 610 differs from the probes 110, 110′ in that the first electrode 300 is selectively moveable. The first electrode 300 can thus be extendible (e.g., to have the configuration of FIG. 3) to facilitate coagulation with the second electrode 302. The first electrode 300 can be retractable such as to the position shown in FIG. 5 to facilitate cutting of tissue using RF energy. More particularly, the first electrode 300 can have at least a portion thereof that is selectively retractable relative to the distal end 612 and the second electrode 302. This retraction can position a first portion of the first electrode 300 back into the shaft 625 such that the first portion of the first electrode 300 can be housed within the shaft 625 proximal of the distal end 612. Only the tip 306 of the first electrode 300 may be exposed when fully retracted as shown in FIG. 5. Retraction of the first electrode 300 can better expose the second electrode 302 for performing cutting of tissue.

FIG. 6 shows the probe 610 need not fully retract the first electrode 300 for cutting purposes. The first electrode 300 is only partially retracted relative to the distal end 612 and the second electrode 302. However, this partial retraction is sufficient retraction to perform cutting of the tissue.

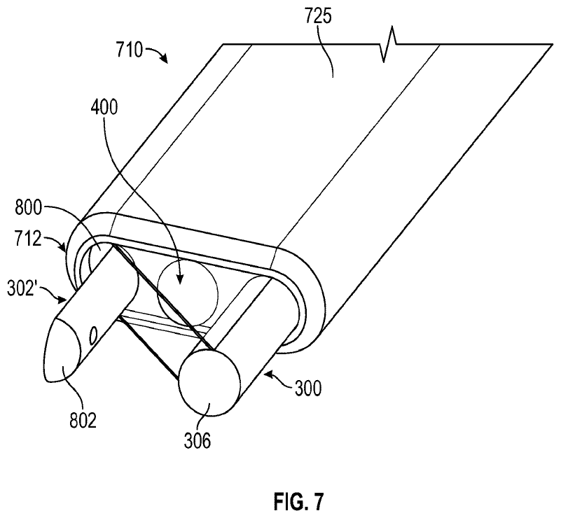

FIG. 7 shows a probe 710 according to another example. The probe 710 is similar to that of the probe 610 in that at least the first electrode 300 is selectively moveable (retractable and extendable). However, the probe 710 can include the apron 400 as previously described in FIG. 4. FIG. 7 illustrates that retraction of the second electrode 302′ can move the apron 400 as well. Such movement can move the apron 400 back distally toward the distal end 712 of the shaft 725. The actuation (movement) of a first portion 800 of the second electrode 302′ can remove the apron 400 from covering a second portion 802 of the second electrode 302′ such that the second portion 802 of the second electrode 302′ is no longer covered by the apron 400. Thus, the second electrode 802 is exposed to the tissue. This second portion is then free to contact the tissue to perform cutting, for example. Thus, the apron 400 is removable from at least a portion of the second electrode 302′ including the tip and/or part of an elongate length thereof with retraction of the first portion of the second electrode 302′. It should be noted that the embodiment of FIGS. 5 and 6 could also be configured to cause actuation to remove an apron in a similar manner with movement of the first electrode into the retracted position pulling the apron back to expose portions of the second electrode for direct contact with the tissue.

It is contemplated that either the first electrode 300 or the second electrode 302′ could be configured to be moveable. FIG. 7 show a configuration where the first portion 802 of the second electrode 302′ is selectively moveable thereby reducing a surface area of the second electrode 302′ exposed to the tissue. The first portion 800 of the second electrode 302′ can represent no less than at least 90%, or 80% or 70% of a total surface area of the second electrode 302′. The second portion 802 of the second electrode 302′ that remains stationary can be no less than at least 10%, 20% or 30% of the total surface area of the second electrode 302′. A surface area of the first electrode 300 can be no more than at least twice as large as a surface area of the second portion 802 of the second electrode 302′.

Various mechanisms can be used to perform actuation of the second electrode 302′ to perform the moving of the second electrode 302′ (or first electrode 300 in the case of the embodiment of FIGS. 5 and 6) as described herein. Examples of these mechanisms are described in the various applications incorporated by reference with the U.S. Application Publications noted above. Such mechanisms can be coupled to the drive mechanism 105 (FIG. 1) to be driven thereby and can include any one or combination of gears, shafts, cams, linear drives, ratchets, collars, springs, etc.

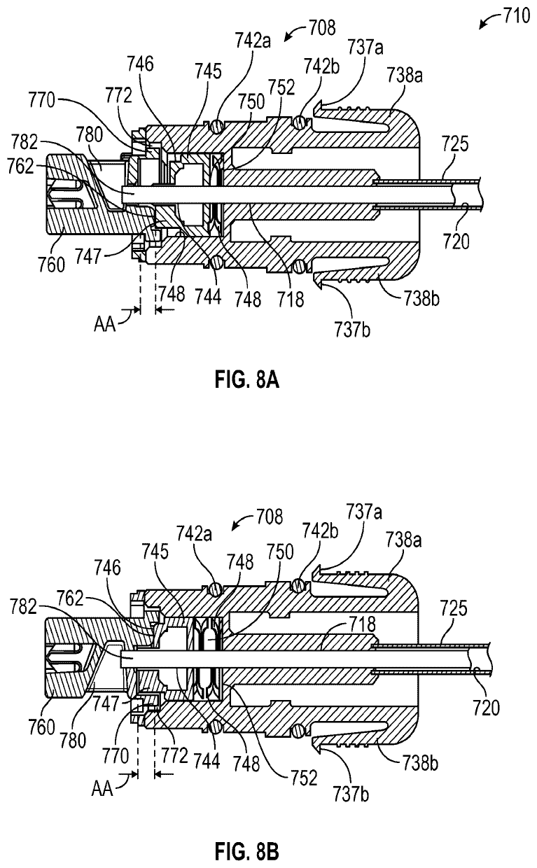

FIGS. 8A and 8B show an example of an actuation mechanism that can axially translate the first electrode in the manner shown in this application. The probe 710 can be locked into the handpiece 104 (FIG. 1) as previously described with regard to probe 110. This can be accomplished by inserting tabs 737a and 737b on flex arms 738a and 738b into receiving openings in the handpiece 104. O-rings 742a and 742b are provided in the hub 708 to seal the hub 708 into the receiving channel in the handpiece 104. The hub 708 can be fixed to the shaft 725. The shaft 725 can comprise an outer sleeve that has multiple channels including channel 720 therein in which a second shaft 718 is slidably disposed. A proximal end 744 of the second shaft 718 has an actuator collar 745 of an electrically conductive material attached thereto with a proximal-facing surface 746 that has a bump or cam surface 747 thereon. The actuator collar 745 can be configured to reciprocate within the bore 748 in the hub 708. FIG. 8A shows the actuator collar 745 in an extended position which corresponds to the extended first electrode position such as shown in FIGS. 1-4B and 9. FIG. 8B shows the actuator collar 745 in a non-extended or retracted position which corresponds to the retracted first electrode position of FIGS. 5-7 and 10.

The actuator collar 745 and hub 708 cam include slot and key features described further below to allow for axial reciprocation of the sliding actuator collar 745 and second shaft 718 while preventing rotation of the collar 745 and the first shaft 725. A spring 748 between a distal surface 750 of actuator collar 745 and a proximally facing internal surface 752 of hub 708 urges the sliding actuator collar 745 and the moveable first electrode toward the retracted or proximal-most position described and shown herein.

Rotating drive coupling 760 can be fabricated of a non-conductive material that rotates in hub 708 as shown in FIGS. 8A and 8B. The drive coupling 760 can have a distal cam surface 762 that engages the proximal-facing cam surface 747 on the actuator collar 745 so that rotation of drive coupling 760 will reciprocate the sliding actuator collar 745 through a forward and backward stroke AA, as schematically shown in FIGS. 8A and 8B. The stroke of the sliding actuator collar 745 and electrode 725 can be between 0.01 mm and 20 mm, and in one variation can be between 0.10 mm and 5 mm. The hub 708 can be provided with a slidable adjustment (not shown) to adjust the distance between the cam surfaces 747 and 762 of the sliding collar 745 and rotating coupling 760, respectively

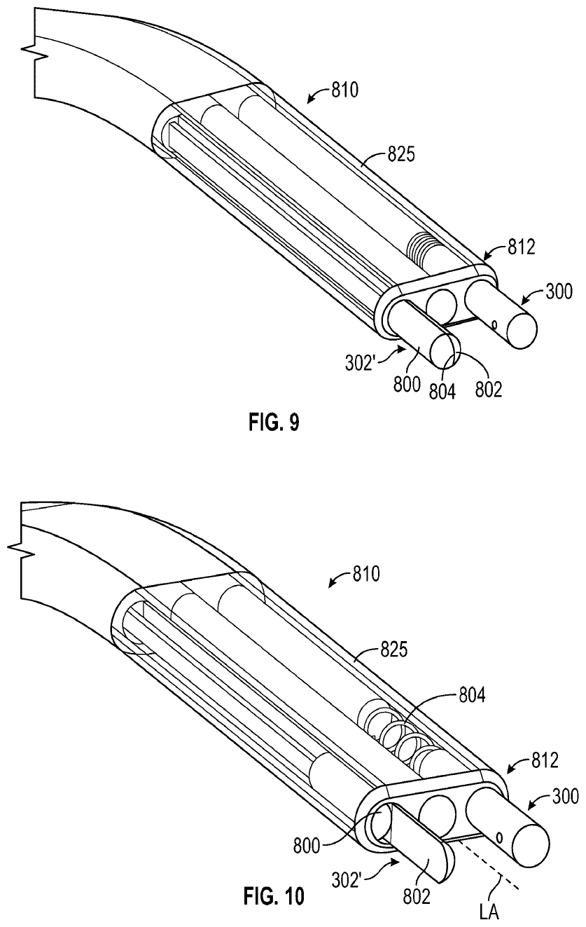

FIGS. 9 and 10 show another example of a distal end 812 of a shaft 825 of a probe 810 according to another embodiment. The probe 810 has a similar construct to that of the probes 110, 110′, 610 and particularly to that of probe 710 of FIG. 7 (save lack of the apron 400 of FIG. 7). The probe 810 has the first portion 800 of the second electrode 302′ is retractable while the second portion 802 is positionally fixed (i.e. is stationary and not retractable with the first portion 800).

The second electrode 302′ in an extended state can have substantially a same shape as the first electrode 300 as shown in the configuration of FIG. 9 (extended position of first portion 800). FIG. 9 can be a position for the second electrode 302′ for performing coagulation. However, the second electrode 302′ can be split along a split line 804. The second portion 802 can be a medial portion while the first portion 800 can be a lateral portion of the second electrode 302′. As shown in FIG. 10, the first portion 800 can be retracted back fully or partially into the shaft 825. The retraction of the first portion 800 leaves the second portion 802, which has a different shape and a different surface area relative to the tissue as compared with the first electrode 300. The first electrode 300 with the larger surface area can act as a return electrode while the second portion 802 of the second electrode 302′ can act as the active electrode. It is noted that the first electrode 300 could also be retracted back into the shaft 825 (such as in the manner of FIGS. 5 and 6) with retraction of the first portion 800 of the second electrode 302′ in FIG. 10. FIG. 10 is a position the electrodes can be used for performing electrosurgical cutting of tissue.

FIG. 9 shows a configuration where a surface area of the first electrode 300 is configured to generate a thermal effect in an electrosurgical coagulation mode and substantially no thermal effect in an electrosurgical resection mode of FIG. 10.

FIG. 10 shows a spring 804 such as a compression spring selectively coupled to the first electrode 300. The spring 804 can be configured to allow the first electrode 300 to be longitudinally movable (along longitudinal axis LA) to contour with the tissue during electrosurgical resection mode of FIG. 10. It should be noted that the first electrode 300 can be stationary during electrosurgical coagulation mode (FIG. 9) and can be selectively movable during the electrosurgical resection mode of FIG. 10. As shown between FIG. 9 and FIG. 10, at least the first portion 800 of the second electrode 302′ can be moveable and movement of the first portion 800 of the second electrode 302′ can engage the first electrode 300 with the spring 804 in the configuration of FIG. 10.

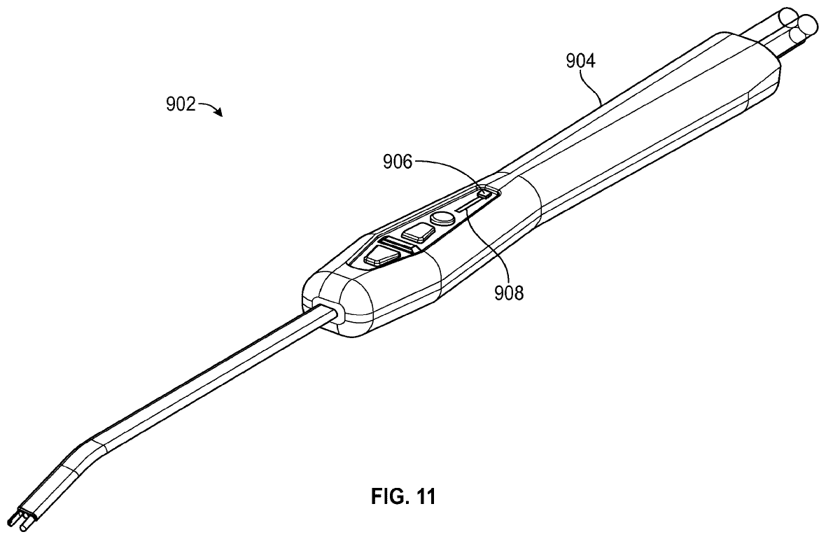

FIG. 11 shows an electrosurgical device 902 similar to those previously described. However, rather than utilizing an electronic button and motor drive to facilitate actuation (movement) of one or more of the electrodes described previously, the handpiece 904 can include a slider mechanism 906 that moves in a slot 908 to drive retraction and extension (selective movement) of the electrode(s).

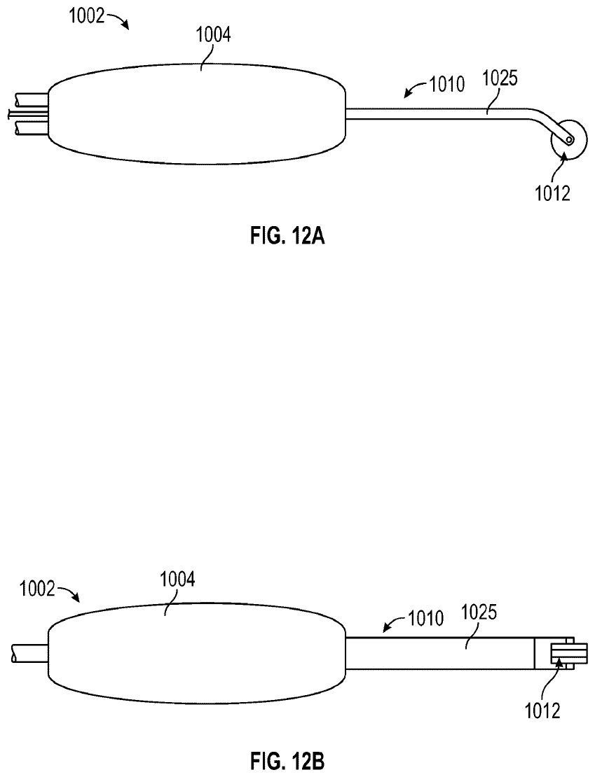

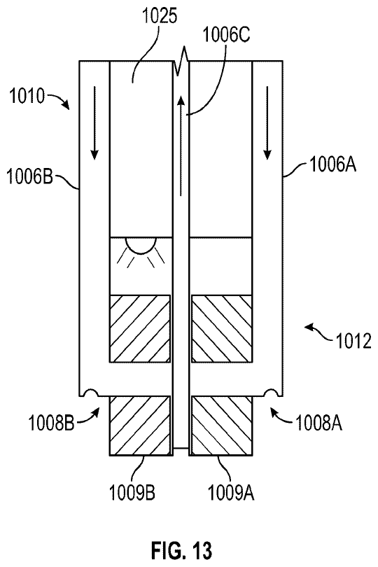

FIGS. 12A-13 show another example of an electrosurgical device 1002 having a handpiece 1004 and probe 1010 similar to those previously described. The probe 1010 can include the shaft 1025 with the distal end 1012.

Turning to FIG. 13, the probe 1010 differs from previous probes in that the probe 1010 can include a plurality of tubes 1006A, 1006B and 1006C extending along the shaft 1025 to the distal end 1012. A first electrode 1008A and the second electrode 1008B can be configured to roll over the tissue with movement of the probe 1010 relative to the tissue. The plurality of tubes 1006A, 1006B and 1006C can be flow channels for the irrigating fluid and aspirated fluid. In particular, the tubes 1006A, 1006B can provide irrigating fluid to tissue adjacent the first electrode 1008A and second electrode 1008B. The tube (third tube) 1006C can provide a flow path for aspirating the fluid (including smoke, tissue debris, etc.) away from the surgical site.

The tubes 1006A and 1006B can also be configured as shafts to support the first electrode 1008A and second electrode 1008B, respectively. However, the tubes 1006A and 1006B need not support the first electrode 1008A and the second electrode 1008B according to further examples. Such support of the first electrode 1008A and the second electrode 1008B could be provided by the shaft 1025 or other components according to further examples.

The first electrode 1008A can be configured as a rolling element (e.g., a wheel). The second electrode 1008B can be configured as a rolling element (e.g., a wheel). The first electrode 1008A can be differently shaped from the second electrode 1008B such as by having a different thickness in a lateral direction. This difference in thickness can facilitate RF current being passed from the first electrode 1008A (active) to the second electrode 1008B (passive).

The first electrode 1008A can be positioned adjacent the distal end 1012. The first electrode 1008A can be rotatably coupled to the shaft 1025 or the first of the plurality of tubes 1006A, for example. The second electrode 1008B can be positioned adjacent the distal end 1012 and spaced from the first electrode 1008A a distance laterally. The second electrode 1008B can be rotatably coupled to one of the shaft 1025 or the second of the plurality of tubes 1006B. The third of the plurality of tubes 1006C can extend distal of the distal end 1012 of the shaft 1025 and can be positioned between the first electrode 1008A and the second electrode 1008B, for example. The third tube 1006C can be positioned a distance from contact surfaces 1009A and 1009B of the first electrode 1008A and the second electrode 1008B with the tissue. This can allow space for gathering the aspirating fluid.

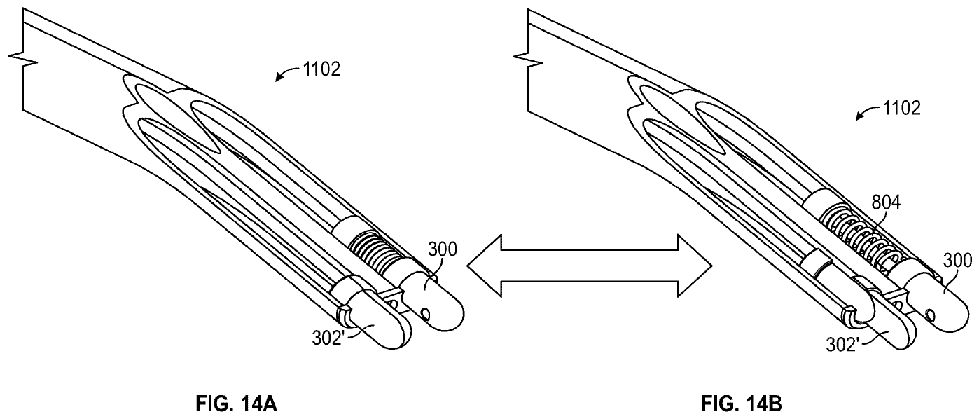

FIGS. 14A and 14B show an example of an electrosurgical device 1102 very similar to the device previously shown and described in FIGS. 9 and 10. Thus, extensive discussion of the configuration of the electrosurgical device 1102 is not provided. As was previously discussed, in coagulation position of FIG. 14A the electrodes 300, 302′ can be of equivalent or substantially a same surface area. This can create a “true bipolar” arrangement of the electrodes 300, 302′.

In the resection position of FIG. 14B, a surface area of the active electrode (here the second electrode 302′) can be reduced by up to 80% as compared to the return electrode (here the first electrode 300). The return electrode 300 can be sprung with the spring 804 so as to freely float and be moveable relative to the distal end as discussed in regard to FIG. 10. Selection of the cutting/ablation mode can increase RF power automatically. Such increase can be between 300 W to 500 W higher from power setting in the coagulation mode of FIG. 14A.

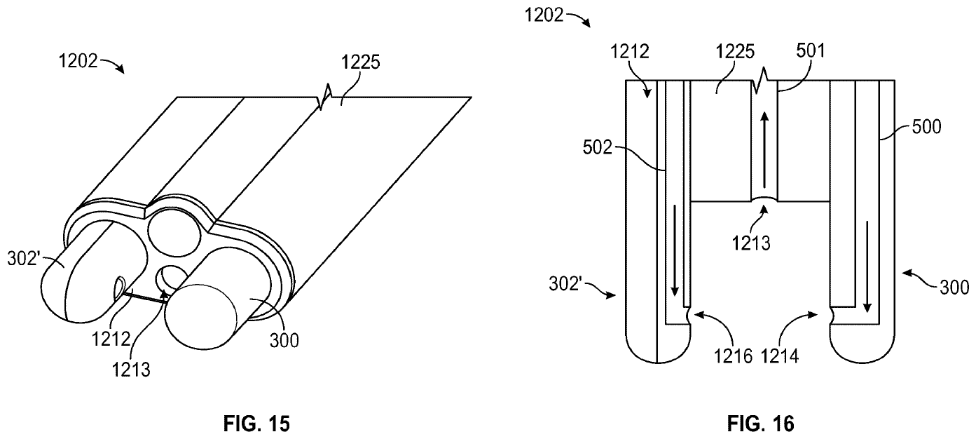

FIGS. 15 and 16 show an electrosurgical device 1202 similar to some of those examples previously described. However, a suction port 1213 has been provided at a distal end 1212 of a shaft 1225 proximal of (and laterally between) the electrodes 300, 302′ and the irrigating fluid outlets. These outlets occur at ports 1214 and 1216, which are provided in the electrodes 300, 302′ as shown in FIG. 16. Thus, a flow passage for the suction no longer passes through one of the electrodes 300, 302′ but is positioned at or adjacent the distal end 1212 proximal of the electrodes 300, 302′.

As with prior examples, the first electrode 300 can be a return electrode and the second electrode 302′ can be an active electrode. The second electrode 302′ can have the second port 1216 therein. The second port 1216 can comprise outlet for irrigating fluid leaving the device from a second flow tube 502 (see FIG. 16 and described previously). The irrigating fluid passes through the second port 1216 and enters the surgical site. The second flow tube 502 defines a flow channel for the irrigating fluid. The second flow tube 502 can be partially defined by the second electrode 302′ according to the example of FIGS. 15 and 16. However, the second flow tube 502 could be a separate component according to further examples. The second flow tube 502 can extend proximally into the shaft 1225 proximal of the distal tip 1212. As discussed previously, the second flow tube 502 can extend along the shaft 1225 and can be in fluid communication with the flow inducing device 226.

The first electrode 300 can have the second port 1214 therein. Differing from prior examples, the first electrode 300 can have the second port 1214 comprise a second inlet for irrigating fluid. The irrigating fluid can pass along a first flow tube 500 (see FIG. 16). The irrigating fluid passes through the port 1214 and enters the surgical site. The first flow tube 500 defines a flow channel for the irrigating fluid. The first flow tube 500 can be partially defined by the first electrode 300 according to the example of FIGS. 15 and 16. However, the first flow tube 500 could be a separate component according to further examples. The first flow tube 500 can extend proximally into the shaft 1225 proximal of the distal tip 1212. As discussed previously, the first flow tube 500 can extend along the shaft 1225 and can be in fluid communication with the flow inducing device 226.

The port 1213 can be an inlet to a flow tube 501 (shown in FIG. 16) that defines a flow channel. The flow tube 501 (also called a flow channel) can be partially defined by the distal tip 1212 and/or the shaft 1225. However, flow tube can be defined by an entirely separate component from the distal tip 1212 and/or shaft 1225 according to some examples. The flow tube 501 can have an inlet (port 1213) substantially between the first electrode 300 and the second electrode 302′, for example. However, the port 1213 can be one of proximal, distal or otherwise offset from the ports 1214 and 1216. The flow tube(s) 501 can extend along the shaft 1225 and can be in fluid communication with the flow channel 224 in handpiece 104 and further in communication with the aspiration tubing 222 which communicates with the negative pressure source 220 (FIG. 1). Via the port 1213, the fluid (e.g., irrigating fluid, blood, tissue debris, smoke, etc.) can be aspirated and pass away from the surgical site via passing through the flow tube 501, etc. The location of the port 1213 in FIGS. 15 and 16 is purely exemplary and can be in other locations as discussed above. The use of additional suction ports in various locations are also contemplated.

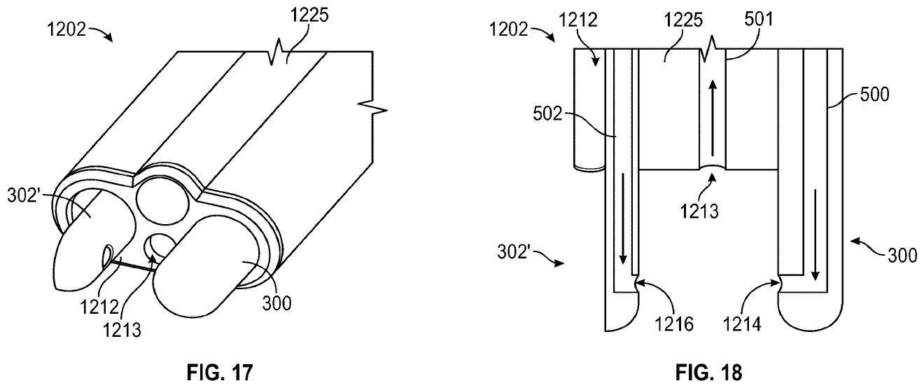

FIGS. 17 and 18 show the electrosurgical device 1202 previously described in FIGS. 15 and 16 operating in an ablation mode. Thus, a portion of the active electrode (the second electrode 302′ is retracted back into the distal tip 1212 to change the surface area of the second electrode 302′ relative to the return electrode (the first electrode 300). The ports 1213, 1214 and 1216 can operate as suction and inlets as previously discussed. RF power can be increased to between 300 W to 500 W from power setting in the coagulation mode of FIGS. 15 and 16.

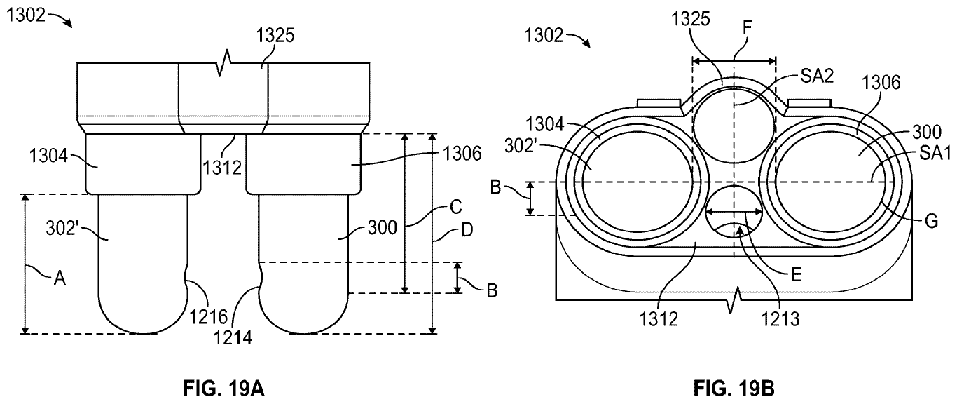

FIGS. 19A and 19B show an electrosurgical device 1302 similar to those previously described including the device 1202 of FIGS. 15-18. The electrosurgical device 1302 can differ in that each of the electrodes 300, 302′ is stood off a distal tip 1312 of a shaft 1325 by a respective boss 1304, 1306. The electrodes 300, 302′ can each include the inflow port 1214 and 1216 (shown in FIG. 19A) as previously described for passing irrigating fluid (saline or other fluid) from the device and into the surgical site. The electrosurgical device 1302 can include the outflow port 1213 (FIG. 19B) as a suction location at the distal tip 1312.

As shown in FIG. 19A, an elongate length of the electrodes 300, 302′ can be between 0.10 inches and 0.35 inches, inclusive, from a distal edge of the respective boss 1304, 1306 (see dimensions A in FIG. 19A). According to one example, a diameter of the port 1214 and/or port 1216 can be between 0.025 inches and 0.055 inches, inclusive (see dimension B in FIG. 19A). A flow rate through the ports 1214 and 1216 can be between 5 ml/min to 100 ml/min, inclusive. A distal most edge of the ports 1214, 1216 can be between 0.125 inches and 0.335 inches, inclusive, from the distal tip 1312 and the port 1213 (see dimension C in FIG. 19A). A distal most tip of the electrodes 300, 302′ can be between 0.15 inches and 0.50 inches, inclusive (see dimension D in FIG. 19A). Thus, the length of the bosses 1304, 1306 can be between 0.05 inches and 0.15 inches, according to some examples. The area of the port 1213 can be substantially the same as the area of the port 1214 or the port 1216 or can be up to about 2 times as large as the area of the port 1214 or port 1216.

Turning to FIG. 19B, the position of the ports 1214 and 1216 is illustrated and is offset from a symmetrical axis SA1 of the electrodes 300, 302′ and the distal tip 1312. Put another way, the location of the ports 1214 and 1216 can be staggered toward a position of the outflow port 1213. The position of the port 1214 and/or port 1216 can extend away from the symmetric axis SA1 to be between 0.025 inches and 0.055 inches, inclusive, from the symmetrical axis SA1 at point of furthest termination. The position of the inflow port 1213 can be staggered from the symmetrical axis SA1 so as to be positioned only on one side thereof.

A diameter of the port 1213 can be between 0.030 inches and 0.110 inches, inclusive (see dimension E in FIG. 19B). A flow rate through the port 1213 can be between 5 ml/min to 600 ml/min, inclusive. A minimum suction flow rate through the port 1213 can be substantially equal to the inflow rate collectively through the ports 1214 and 1216. However, the suction flow rate through the port 1213 can be anywhere from 1.1:1 to 6:1 times greater as compared to the inflow rate collectively through the ports 1214 and 1216. Inflow and suction flow rates are paired to create a stable fluid equilibrium at the surgical site. The flow rate through the port 1213 can be about 3:1 times greater than the inflow rate collectively through the ports 1214 and 1216 according to some examples. The electrodes 300, 302′ can be spaced apart a distance of 0.05 inches and 0.15 inches, inclusive (see dimension F). The electrodes 300, 302′, and hence the ports 1214 and 1216, can be symmetrically arranged with respect to a second symmetric axis SA2. A diameter of the electrodes 300, 302′ can be between 0.075 inches and 0.20 inches, inclusive (see dimension G in FIG. 19B).

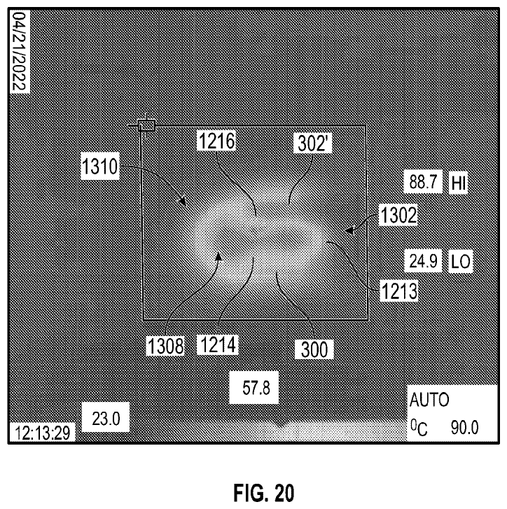

FIG. 20 shows the electrosurgical device 1302 operating to inflow irrigating fluid 1308 (or other liquid) to a surgical site 1310. The electrodes 300, 302′ can be seen. FIG. 20 shows a heat map of the surgical site 1310 and the irrigating fluid 1308. The irrigating fluid 1308 can flow to within only a relatively small area of the surgical site 1310. Irrigating fluid 1308 spread (area within the surgical site 1310) can be controlled with the inflow from the ports 1314, 1316 and/or outflow of the irrigating fluid 1308 at the port 1213 (see discussion of flow rates and relative flow rates above). The irrigating fluid 1308 can be kept within less than about 2 inches of the distal tip of the electrodes 300, 302′, for example. Control of irrigating fluid 1308 inflow and suction results in targeted tissue coagulation without unintended collateral damage to tissue do to overheating. A peak temperature of the irrigating fluid 1308 can be 71° C., which can avoid unintended collateral damage to tissue do to overheating. Furthermore, because irrigating fluid 1308 inflow and suction is tightly controlled allowing irrigating fluid 1308 to only a relatively small area of the surgical site 1310, RF power levels can be kept lower while still achieving equivalent coagulation performance.

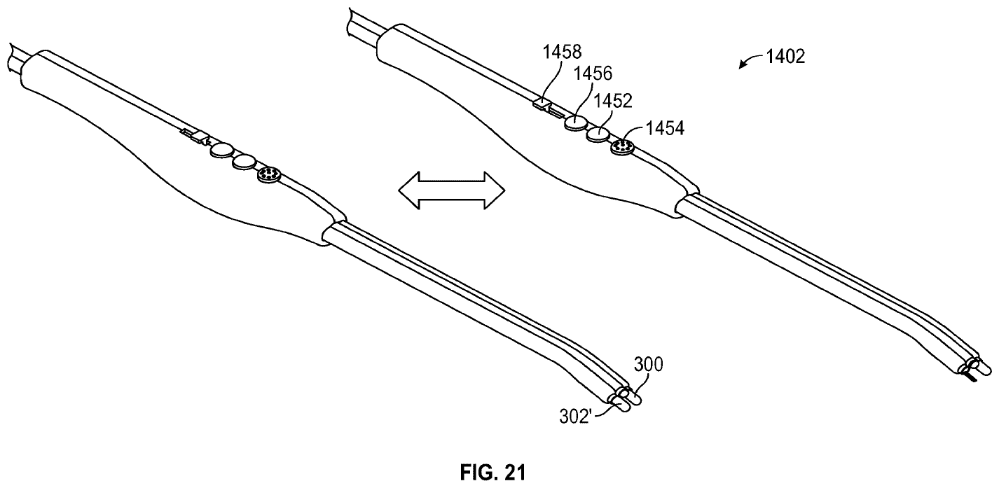

FIG. 21 shows an example of an electrosurgical device 1402 very similar to the devices previously shown and described in FIGS. 9, 10, 14A, 14B, 15-18, 19A and 19B. Thus, extensive discussion of the configuration of the electrosurgical device 1402 is not provided but discussion is focused on features or components that have been altered or added. As was previously discussed, in the coagulation position of FIG. 21 to the viewer's left the electrodes 300, 302′ can be of equivalent or substantially a same surface area (e.g., the active electrode can have a surface area of 0.1036 in² while the return electrode can have a surface area of 0.104 in² for an area ratio of 1.004:1.0). This can create a “true bipolar” arrangement of the electrodes 300, 302′.

The electrosurgical device 1402 can have dedicated buttons for setting and/or changing operation modes (e.g., coagulation power output button 1452, ablation power output button 1454, Light activation button 1456, slider 1458—allows surgeon to between the two electrode configurations available (Cut (viewer's right) and Coagulation (viewer's left)) by simply sliding this component). Handle and other features including the probe of the electrosurgical device 1402 can be disposable after surgery according to one example.

In the resection position to the viewer's right in FIG. 21, a surface area of the active electrode (here the second electrode 302′) can be reduced by 80% or more (e.g., up to much as 95%) as compared to the return electrode (here the first electrode 300). The return electrode 300 can be sprung so as to freely float and be moveable relative to the distal end as previously discussed. Selection of the cutting/ablation mode can increase RF power automatically or via toggling the ablation power output button 1454. Such increase can be between 300 W to 500 W higher from power setting in the coagulation mode.

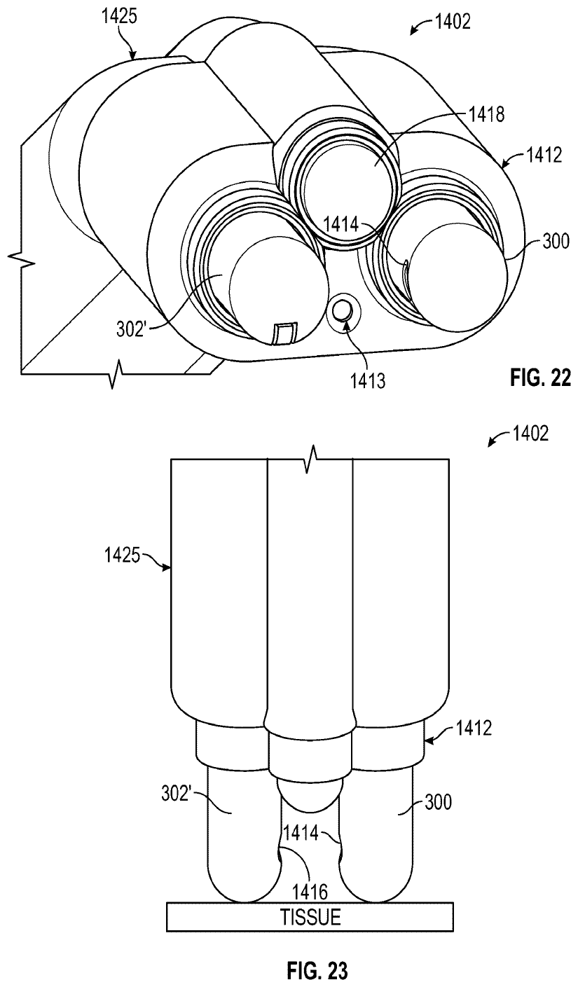

FIGS. 22-24 show the electrosurgical device 1402 in the coagulation mode setting. A suction port 1413 has been provided at a distal tip 1412 of a shaft 1425 proximal of (and laterally between) the electrodes 300, 302′ and the irrigating fluid outlets similar to the example of FIGS. 15-19B. Thus, an illumination element 1418 can be included with the device 1402 (FIG. 22). Outlets for saline or other irrigating fluid occur at ports 1414 and 1416 (FIG. 23), which are provided in the electrodes 300, 302. Thus, a flow passage for the suction no longer passes through one of the electrodes 300, 302′ but is positioned at or adjacent the distal tip 1412 proximal of the electrodes 300, 302′.

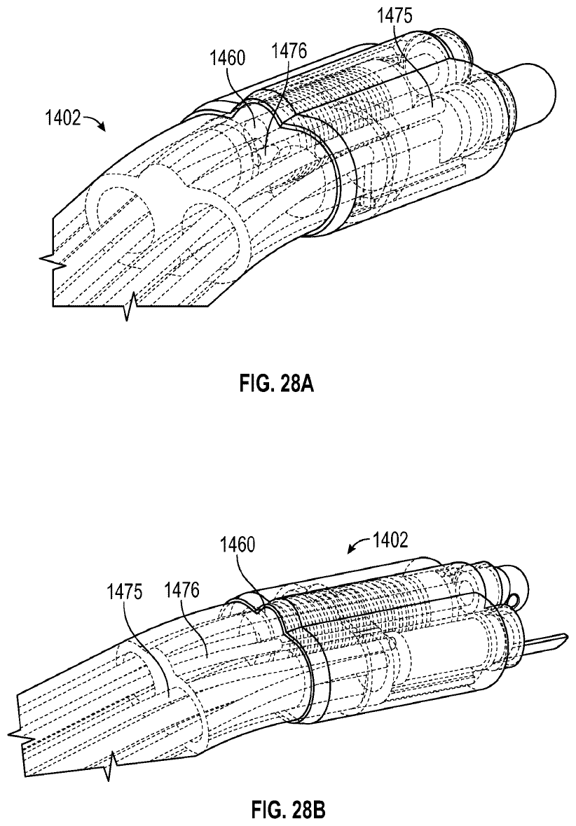

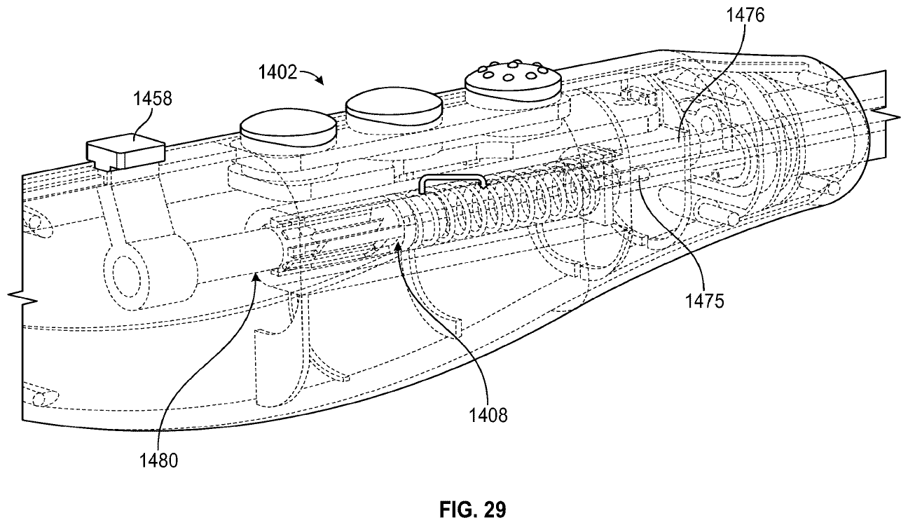

As with prior examples, the first electrode 300 can be a return electrode and the second electrode 302′ can be an active electrode. The second electrode 302′ can have the second port 1416 therein (FIG. 23). The second port 1416 can comprise outlet for irrigating fluid leaving the device from a second flow tube (see FIG. 16 described previously and further in FIGS. 28A-29B). The irrigating fluid passes through the second port 1416 and enters the surgical site. The first electrode 300 can have the second port 1414 therein. The first electrode 300 can have the second port 1414 comprise a second inlet for irrigating fluid. The irrigating fluid can pass along a first flow tube 500 (see FIG. 16 described previously and further in FIGS. 28A-29B). The irrigating fluid passes through the port 1414 and enters the surgical site. The flow rate of the irrigating fluid can be between about 9 ml/min to about 100 ml/min and can be controlled as desired.

The port 1413 can be an inlet to a flow tube and can be partially defined by the distal tip 1412 and/or the shaft 1425. However, flow tube can be defined by an entirely separate component from the distal tip 1412 and/or shaft 1425 according to some examples. The inlet (port 1413) can be substantially between the first electrode 300 and the second electrode 302′, for example. The port 1413 can have a diameter of between about 0.025 inches 0.050 inches, for example. According to further examples, the port 1413 can be one of proximal, distal or otherwise offset from the ports 1414 and 1416. As shown in FIG. 22, the port 1413 is distal of most or all of the ports 1414 and 1416 and the first electrode 300 and the second electrode 302′. The flow tube(s) can extend along the shaft 1425 and can be in fluid communication with the flow channel 224 (discussed previously) in handpiece and further in communication with the aspiration tubing which communicates with the negative pressure source (FIG. 1). Via the port 1413, the fluid (e.g., irrigating fluid, blood, tissue debris, smoke, etc.) can be aspirated and pass away from the surgical site via passing through the flow tube, etc. The flow rate of such fluid can be between about 9 ml/min to about 100 ml/min and can be controlled as desired.

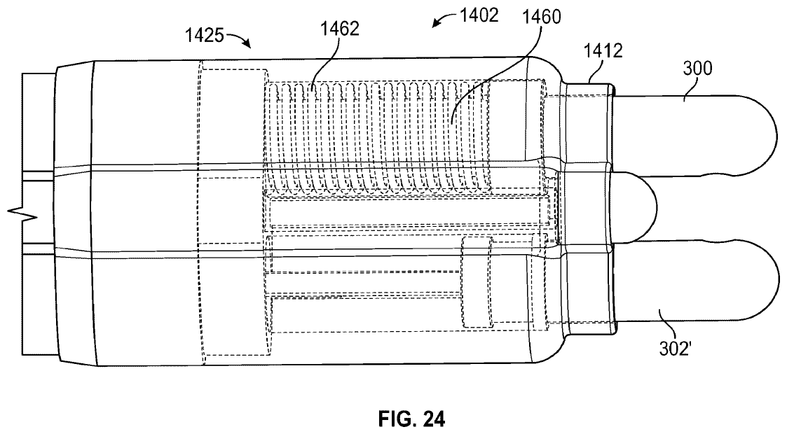

FIG. 24 illustrates a portion of the shaft 1425 and distal tip 1412 in phantom to show a positioning mechanism 1460 internal to the shaft 1425 fitted between a spring 1462. The positioning mechanism 1460 actuated via the slider 1458 (FIG. 21) can be used to fix the position of the return electrode 300 relative to the distal tip 1412 in a protruded state during coagulation mode. The diameter of the first electrode 300 and the second electrode 302′ can be between about 0.0075 inches and about 0.020 inches, for example. The distance the first electrode 300 and the second electrode 302′ extend distal of the distal tip 1412 can be between about 0.20 inches and about 0.30 inches, for example. The spacing between the innermost edge of the first electrode and the innermost edge of the second electrode 302′ can be between about 0.075 inches and about 0.125 inches, for example.

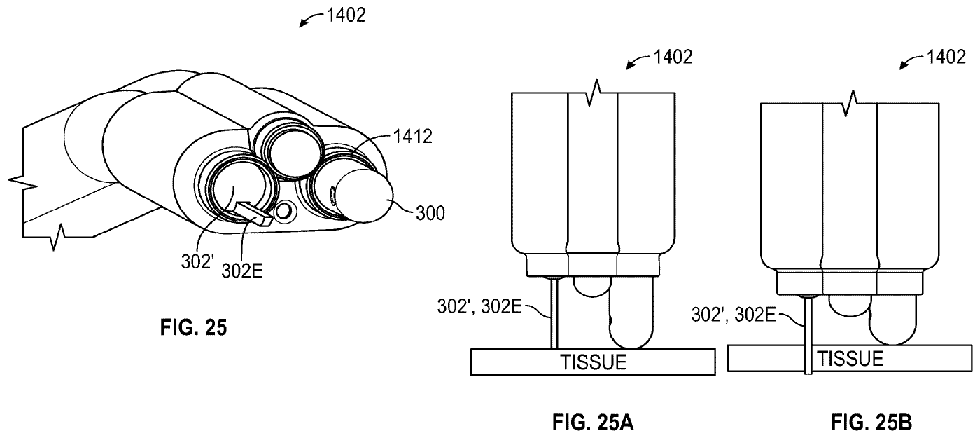

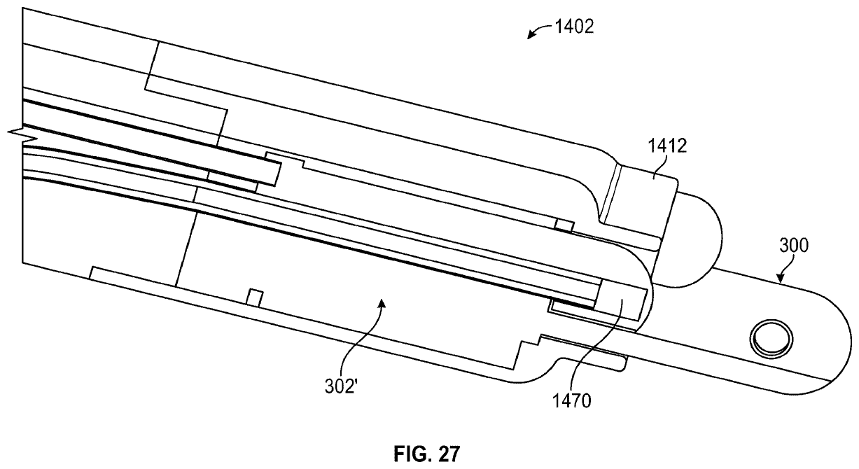

FIGS. 25-27 show the electrosurgical device 1402 previously described in FIGS. 21-24 operating in the ablation mode. Thus, a portion of the active electrode (the second electrode 302′ is retracted back into the distal tip 1412 to change the surface area of the second electrode 302′ relative to the return electrode (the first electrode 300). The ports 1413, 1414 can operate as suction and inlet as previously discussed. However, as shown in the cross-section of FIG. 27, the port 1416 of the active electrode 302′ can be blocked by a feature 1470 of the shaft 1425 or the distal tip 1412. This feature 1470 can be a silicone or other material. This feature 1470 can block flow from the active electrode 302′. Thus, only one inlet for irrigating fluid on the return electrode 300 is used with the device 1402. RF power can be increased in the ablation mode by use of the ablation power output button 1454 (FIG. 21) to between 300 W to 500 W from power setting in the coagulation mode.

As shown in FIG. 25, most of the second electrode 302′ is retracted into the distal tip 1412 leaving only a centrally and distally positioned extending portion 302E of the second electrode 302′ extending distally of the distal tip 1412. This extending portion 302E can have a height of between about 0.020 inches and about 0.040 inches and can have a thickness of between about 0.010 inches and about. 0.030 inches. The surface area of the extending portion of the second electrode 302′ can between 0.010 and 0.030 inches², for example. In contrast, the surface area of the first electrode 300 can be between about 0.09 inches and about 0.12 inches, for example. The distance the second electrode 302′ (the extending portion 302E) extends distal of the distal tip 1412 can remain between about 0.20 inches and about 0.30 inches, for example.

FIG. 25A shows initial contact of the first electrode 300 and the second electrode 302′ via the extending portion 302E with tissue and cutting of the tissue (FIG. 25B) with the second electrode 302′. As an example, initial contact of FIG. 25A can only be along outer surface and distal tip of the extending portion 302E of the second electrode 302′ prior to full cutting contact along the sides and other surfaces of the extending portion of the second electrode 302′.

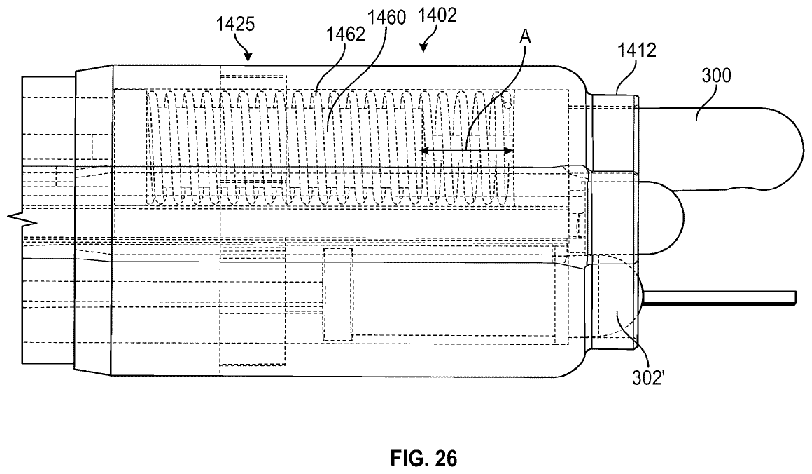

FIG. 26 illustrates a portion of the shaft 1425 and distal tip 1412 in phantom to show the positioning mechanism 1460 internal to the shaft 1425 disengaged allowing for movement of that positioning mechanism 1460 and the first electrode 300 as indicated by arrow A. A bias of the spring 1462 can be used to position of the return electrode 300 relative to the distal tip 1412. This allows the return electrode 300 to be moveable (e.g., toward or away from the distal tip 1412) during the ablation mode.

FIGS. 28A and 28B show actuation of the second electrode 302′ and the positioning mechanism 1460 associated with the first electrode 300 via push rod 1475 and push rod 1476, respectively. FIG. 28A shows the device 1402 in the coagulation mode. FIG. 28B shows the device 1402 in the ablation mode. The positioning mechanism 1460 can have a through hole to accommodate the tube feeding the irrigating fluid to the first electrode 300. This inlet flow tube can stay fixed to the first electrode 300 while the positioning mechanism 1460 can be fixed to stay stationary during coagulation mode discussed previously. Alternatively, inlet flow tube can stay fixed to the first electrode 300 while the positioning mechanism 1460 can free to ride up and down relative to the inlet flow tube during the ablation mode discussed previously.

FIG. 29 shows further features of the electrosurgical device 1402 including an actuator mechanism 1480 that includes a hub 1408 internal to the electrosurgical device 1402. The hub 1408 can be coupled to the slider 1458 and selectively to the push rod 1475 and/or push rod 1476. The actuator mechanism 1480 can configured like a actuator pen or other feature and can include a spring and locking mechanism that are part of the hub 1408. The hub 1408 can be configured depending upon the position of the slider 1458 to couple with one of the push rod 1475 or the push rod 1476.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The term “substantially”, “generally” or “about” mean within 15% of the value provided. The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

1. A probe for an electrosurgical device for treating tissue, the probe comprising:
an elongated shaft having a proximal end, a distal end, and a longitudinal axis;
a first electrode selectively extending from the distal end of the shaft; and
a second electrode selectively extending from the distal end of the shaft, wherein the second electrode is spaced from the first electrode.

2.-6. (canceled)

7. The probe of claim 1, further comprising a plurality of tubes extending along the shaft to the distal end, wherein the first electrode is connected to at least a first of the plurality of tubes and has a channel therein extending to a first port defined by the first electrode, and wherein the second electrode is connected to at least a second of the plurality of tubes and has a channel therein extending to a second port defined by the second electrode.

8. The probe of claim 1, wherein at least a first portion of the second electrode is retractable relative to one or both of the first electrode and a second portion of the second electrode.

9. The probe of claim 8, wherein the first portion of the second electrode is longitudinally retractable into the shaft.

10. (canceled)

11. The probe of claim 8, wherein retraction of the first portion of the second electrode changes a surface area of the second electrode exposed to the tissue.

12. The probe of claim 1, wherein one of the first electrode has a different shape from the second electrode or the first electrode has a substantially identical shape as the second electrode.

13. The probe of claim 1, further comprising an illumination device positioned at the distal end of the shaft.

14.-16. (canceled)

17. The probe of claim 1, wherein the first electrode and the second electrode have a first configuration relative to one another in an electrosurgical coagulation mode.

18. The probe of claim 17, wherein the first electrode and the second electrode have a second configuration relative to one another in an electrosurgical resection mode.

19. (canceled)

20. The probe of claim 1, wherein a first portion of the second electrode is selectively moveable thereby reducing a surface area of the second electrode exposed to the tissue.

21. The probe of claim 20, wherein the first portion of the second electrode represents no less than at least 90%, or 80% or 70% of a total surface area of the second electrode.

22. The probe of claim 21, wherein a second portion of the second electrode that remains stationary is no less than at least 10%, 20% or 30% of the total surface area of the second electrode.

23. The probe of claim 22, wherein a surface area of the first electrode is no more than at least twice as large as a surface area of the second portion of the second electrode.

24.-35. (canceled)

36. A probe for an electrosurgical device for treating tissue, the probe comprising:
an elongated shaft having a proximal end, a distal end, and a longitudinal axis;
a first electrode selectively extending from the distal end of the shaft, the first electrode defining a first inflow port for an irrigating fluid;
a second electrode selectively extending from the distal end of the shaft, wherein the second electrode is spaced from the first electrode, and wherein the second electrode defines a second inflow port for the irrigating fluid; and
an outflow port positioned between the first electrode and the second electrode and spaced a distance from the first inflow port and the second inflow port, wherein the outflow port is configured to receive the irrigating fluid.

37. The probe of claim 36, further comprising a plurality of tubes extending along the shaft to the distal end, wherein the first electrode is connected to at least a first of the plurality of tubes and has a channel therein extending to the first inflow port defined by the first electrode, wherein the second electrode is connected to at least a second of the plurality of tubes and has a channel therein extending to the second inflow port defined by the second electrode, and wherein a third of the plurality of tubes has a channel therein extending to communicate with the outflow port.

38. The probe of claim 36, wherein a flow rate of the irrigating fluid through the outflow port is between 1.1 times and 6 times greater than a flow rate of the irrigating fluid through the first inflow port and the second inflow port collectively.

39. (canceled)

40. The probe of claim 38, wherein the flow rate of the irrigating fluid through the outflow port is between 5 ml/min to 600 ml/min, inclusive, and the flow rate of the irrigating fluid through the first inflow port and the second inflow port collectively is between 5 ml/min to 100 ml/min, inclusive.

41. The probe of claim 36, wherein a distal most edge of the first inflow port and the second inflow port is between 0.125 inches and 0.335 inches, inclusive, from the outflow port.

42. The probe of claim 36, wherein an area of the outflow port is substantially the same as an area of the first inflow port or the second inflow port or can be up to about 2 times as large as the area of the first inflow port or the second inflow port.

43. The probe of claim 36, further comprising a boss configured to cover a portion of one of the first electrode or the second electrode, wherein the boss extends distally from the distal end of the elongate shaft.
